(19) United States
(12) Patent Application Publication
(10) Pub. No.: US 2015/0035208 A1
(43) Pub. Date: Feb. 5, 2015

(54) SEALING MEMBER FOR VACUUM SUSPENSION PROSTHETIC DEVICE

(71) Applicant: Ralph E. BERNAQUER, JR., Ellsworth, ME (US)

(72) Inventor: Ralph E. BERNAQUER, JR., Ellsworth, ME (US)

(21) Appl. No.: 14/516,780

(22) Filed: Oct. 17, 2014

Related U.S. Application Data
(62) Division of application No. 13/495,128, filed on Jun. 13, 2012, now abandoned.

(51) Int. Cl. B29C 39/42; A61F 2/78; A61F 2/80

(52) U.S. Cl. CPC B29C 39/42; A61F 2/80; A61F 2/7812; A61F 2002/807; B29K 2019/00
USPC 264/571

(57) ABSTRACT

A method for creating an improved sealing member for use with a vacuum suspension prosthetic device, with the sealing member located along the top edge of a stump socket and made of a pliable material to provide an improved seal between the stump and the socket in order to maintain vacuum levels within the socket and to minimize cutting or tearing of the stump liner.

### CROSS REFERENCE TO RELATED APPLICATION

This application is a divisional of U.S. Ser. No. 13/495,128, filed Jun. 13, 2012 and currently pending, entitled Sealing Member For Vacuum Suspension Prosthetic Device, by Ralph E. Bernaquer, which is hereby incorporated by reference.

### BACKGROUND OF THE INVENTION

1. Technical Field

The present invention relates generally to the field of prosthetic devices. More particularly, the present invention is directed to a sealing member capable of providing an improved seal for maintaining a vacuum in a vacuum suspension system used for attaching a prosthetic leg to an amputee's residual stump.

2. Description of Prior Art

Prosthetic devices have been a boon to persons who have lost limbs, either to disease or through traumatic injury. Though prosthetic limbs do not replace all of the functionality of the lost limb, they allow the wearer to accomplish much, if not all, of the tasks associated with a natural limb.

Prosthetic limb technology has evolved over time, from extremely simple devices to highly sophisticated devices. With regard to prosthetic legs, one recent development has been the vacuum suspension system.

A vacuum suspension system works with a prosthetic device that is comprised of a socket and a prosthetic leg. The socket is an attachment member which provides an interface between the wearer's residual stump and the prosthetic leg. It is typically a rigid container having an open top end and an interior, whereby the stump is placed into the interior of the socket through the open top end. The bottom exterior of the socket comprises mechanisms to attach the prosthetic leg. There are typically one or more liners used with the socket, to protect the stump and to increase the comfort level to the wearer when the socket is being worn. The efficiency of a socket is dictated by the integrity of the fit between the stump and the socket; a socket that fits securely, with a minimum of movement between the stump and socket, will allow the wearer to most effectively use the prosthetic leg. However, where the fit between the stump and the socket becomes loose or otherwise unstable, the wearer is unable to use the prosthetic leg to its fullest capabilities, resulting in difficulty in ambulating, increased fatigue, increased danger of falls, decreased comfort, and increased risk of injury to the stump.

In a vacuum suspension system, the vacuum suspension component consists of a vacuum pump that pumps air out of the socket when it is being worn, creating a vacuum. The vacuum secures the stump in place within the socket. An airtight socket is critical for success with vacuum suspension systems. In order to create the vacuum, a special rubberized stump liner is used. The stump liner is placed over the stump before the stump is placed into the socket. During use, the upper portion of the stump liner extends above the top of the socket while the lower portion resides within the interior of the socket. The stump liner is trapped between the stump and the top edge of the socket, creating a lower seal. Any air between the stump liner and the interior surface of the socket is evacuated by the vacuum pump, creating the vacuum that secures the socket to the stump. An external socket sleeve is also used, placed over the top part of the socket and the portion of the stump located outside of the socket, such that the socket sleeve covers the top edge of the socket. The top portion of the stump liner is rolled down such that the top portion of the socket sleeve makes contact with the inner part of the top portion of the stump liner. This contact creates a second seal. An example of a vacuum suspension system is the LimbLogic® VS, made by the Ohio Willow Wood Company.

However, through normal use, leaks develop allowing air to escape the socket, thereby breaking the vacuum. Once the vacuum is broken the integrity of the fit of the socket is compromised. To account for this, vacuum suspension systems also incorporate sensors which continuously monitor the level of the vacuum. As soon as a decrease in the level of the vacuum below a preset level is detected, the vacuum pump is activated and restores the vacuum to the proper level. The wearer may adjust the preset vacuum level for comfort and performance.

While vacuum suspension systems have caused prosthetic legs to become far more useful to the wearer, greatly improving the range of functionality of the prosthetic leg, current systems have their drawbacks. The primary drawback of any vacuum suspension system is the inability of the socket to maintain a constant appropriate level of vacuum during use. Because of frequent leakage, the vacuum pump must run on a relatively constant basis. This severely limits the time that the prosthetic device can be used before the pump's power supply (typically a rechargeable battery) is depleted. This also presents some degree of danger, if the power supply for the pump becomes depleted while the wearer is in a location where replacement or recharging is not practical. In such situations the prosthetic leg may be rendered useless. Even when there is ample power, the constant operation of the pump causes an annoying noise as well as fluctuating pressures on the stump.

Another undesirable consequence of insufficient vacuum integrity is increased wear and tear on the stump liner. Each time the socket leaks and the vacuum is broken, the fit of the socket on the stump becomes looser and the socket moves relative to the stump. Even slight movements cause frictional rubbing between the top edge of the socket and the stump liner. If the stump liner becomes cut or torn from such rubbing it will no longer hold a seal and will need to be replaced. Stump liners typically have a useful life of only a few weeks.

Yet another undesirable consequence of insufficient vacuum integrity is increased trauma to the stump. Movement of the socket relative to the stump due to loss of vacuum irritates the skin. Itching and excess sweat may result. In severe cases the hairs of the stump may be rubbed off or become ingrown, sores may develop, or the stump may become too tender to bear a socket.

It is evident that there is a need for an improved sealing member for vacuum suspension systems.

It is therefore an object of the present invention to provide a method for creating an improved sealing member for vacuum suspension systems.

It is another object of the present invention to provide a method for creating an improved sealing member for vacuum suspension systems that reduces the incidence of leakage, thereby maintaining the vacuum at desired levels for greater lengths of time.

It is yet another object of the present invention to provide a method for creating an improved sealing member for vacuum suspension systems that allows for extended battery life for the vacuum pump.

It is yet another object of the present invention to provide a method for creating an improved sealing member for vacuum suspension systems that reduces the incidence of stump liner cuts.

It is yet another object of the present invention to provide a method for creating an improved sealing member for vacuum suspension systems that reduces the trauma to the stump caused by the socket, thereby improving stump health.

Other objects of the present invention will be readily apparent from the description that follows.

### SUMMARY OF THE INVENTION

The present invention comprises an improved sealing member for a vacuum suspension prosthetic device, whereby the sealing member is placed along the top edge of the socket opening. The sealing member is designed to protect the stump liner from the sharp rigid top edge of the socket. It is also designed to provide a more efficient seal between the socket and the stump liner, thereby improving the ability of the vacuum suspension prosthetic device to maintain a vacuum. The sealing member is made of a pliable, nonfriable material, thereby being somewhat deformable when pressed against by the residual stump. This property of the sealing member allows a small amount of “give” during movement, thereby preventing gapping that could otherwise occur and cause leaks, though still providing a firm region of contact to maintain stability and control during use. Moreover, the sealing member does not present a sharp rigid surface to the stump liner, thereby minimizing tearing or cutting of the stump liner. The sealing member may be integrated with the top edge of the socket or affixed thereto.

The present invention also discloses methods for retrofitting a socket with the improved sealing member. This may be accomplished by placing a pre-formed sealing member over the top edge of a socket. It may also be accomplished by applying layers of resilient material to the top edge of the socket to build up a sealing member.

Other features and advantages of the present invention are described below.

### DESCRIPTION OF THE DRAWINGS

FIG. 1 depicts a prior art configuration, showing a residual stump, a stump liner, a socket sleeve, and a socket.

FIG. 2 depicts the configuration shown in FIG. 1, with the residual stump placed into the stump liner and then placed into the socket and the socket sleeve pulled over the socket and the stump liner.

FIG. 3 depicts an embodiment of the present invention whereby the socket is viewed in perspective and the sealing member is formed onto and affixed to its top edge.

FIG. 4 depicts the embodiment of the present invention shown in FIG. 3, with the residual stump placed into the stump liner and then placed into the socket (the socket sleeve is shown cut-away to provide clearer detail of the sealing member).

FIG. 5 depicts an alternative embodiment of the present invention whereby the socket is viewed in perspective and a pre-formed sealing member is shown ready to be placed onto the top edge of the socket.

### DETAILED DESCRIPTION OF INVENTION

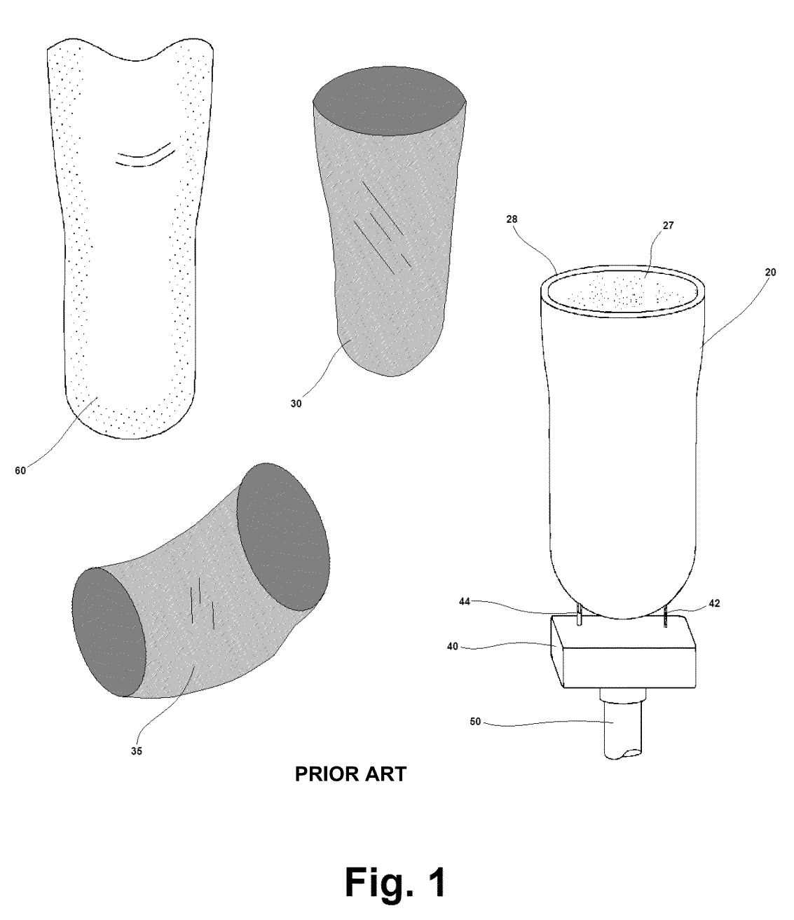

The present invention discloses an improved sealing member 10 to be used with a vacuum suspension prosthetic device. The vacuum suspension prosthetic device has the following components: a socket 20, a stump liner 30, a socket sleeve 35, a vacuum pump 40 with a vacuum level sensor 42, and a prosthetic leg 50. See FIG. 1. The socket 20 serves as the adapter between the residual stump 60 and the prosthetic leg 50. It is a rigid container having substantially continuous side and bottom walls and inner and outer surfaces 24,25. Its open top end 27 is defined by a top edge 28. The interior space 29 of the socket 20 is configured to receive the residual stump 60. On the outer surface 25 of the socket 20, at the bottom of the socket 20, are the attachment means used to secure the prosthetic leg 50 to the socket 20.

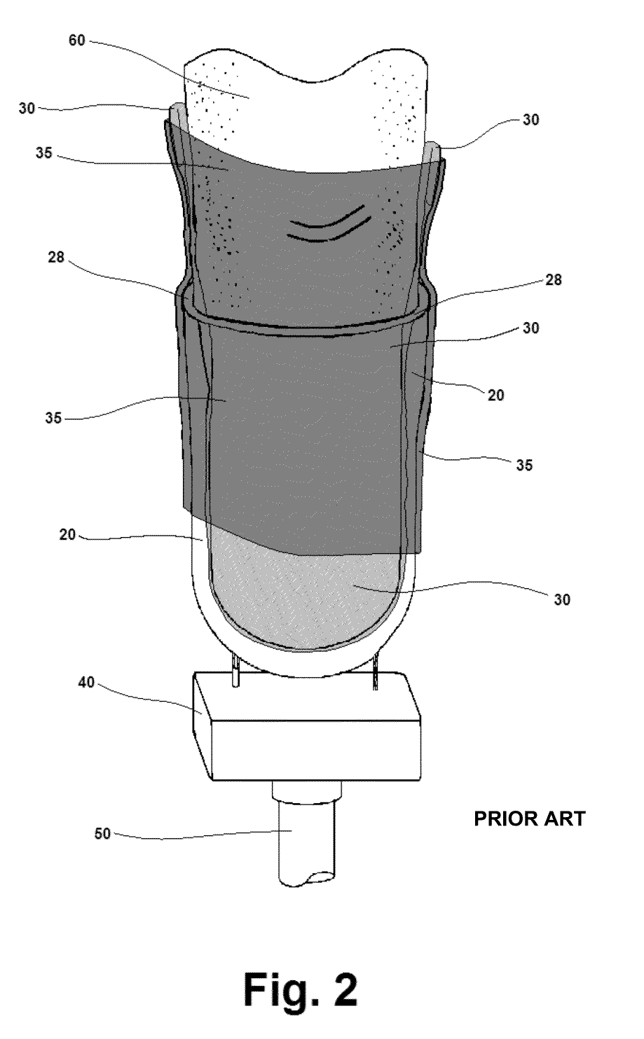

The stump liner 30 is used in conjunction with the socket 20 and is placed over the residual stump 60. It is a flexible sleeve having an open top end and a closed bottom end, and fits snugly over the residual stump 60. It is substantially air impermeable. When placed over the residual stump 60 and then placed into the socket 20, the stump liner 30 presses against the inner surface 24 of the socket 20. See FIG. 2.

The socket sleeve 35 is placed over the socket 20 and over the stump 60. It is a flexible sleeve having open top and bottom ends and fits snugly over the socket 20. It is substantially air impermeable. After the residual stump 60 and stump liner 30 are placed into the socket 20, the top portion of the socket sleeve 35 is rolled up against the top portion of the stump liner 30 that extends out of the socket 20 and which is folded down. See FIG. 2. The top portions of the socket sleeve 35 and the stump liner 30 form an air impermeable barrier. See FIG. 2.

The vacuum pump 40 is a device capable of evacuating air from the interior space 29 of the socket 20 to create at least a partial vacuum therein. It is in communication with the interior space 29 of the socket 20, typically through an air valve 44. The vacuum level sensor 42 is in communication with the interior space 29 of the socket 20. It is capable of ascertaining the vacuum level within the interior space 29 of the socket 20. It is also in communication with the vacuum pump 40. Depending on the vacuum level ascertained by the vacuum level sensor 42, operation of the vacuum pump 40 is either engaged to increase or reestablish at least a partial vacuum within the socket 20 or rendered inoperable because an appropriate vacuum level is present.

The sealing member 10 of the present invention is constructed of a pliable material. The material should be nonfriable, resilient, and durable. In some embodiments the material comprising the sealing member 10 may have a liquid state and a solid state. An example of such a material is synthetic rubber, such as Performix® Brand Plasti Dip®. Other materials may also be used, such as closed cell foam and natural rubber.

The sealing member 10 should have a substantially uniform height and a substantially uniform thickness. It has a top side 12, an outer lateral side 14, and an inner lateral side 16. It is substantially rounded or curved where its top side 12 transitions to its inner lateral side 16, thereby avoiding any hard edges on its inner side 16.

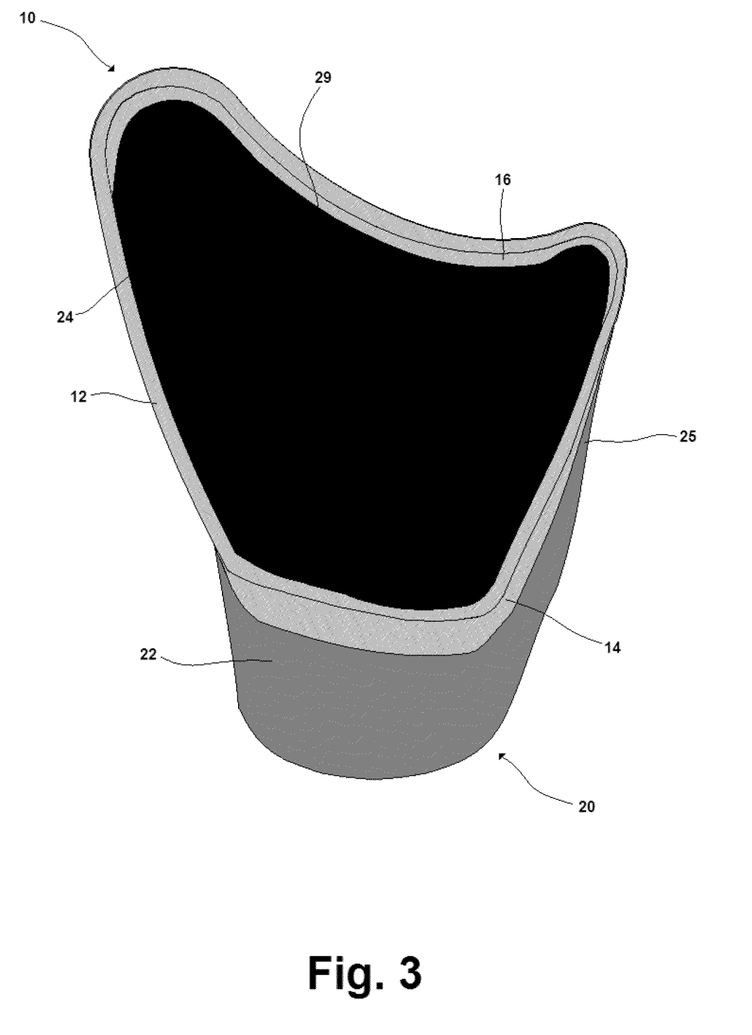

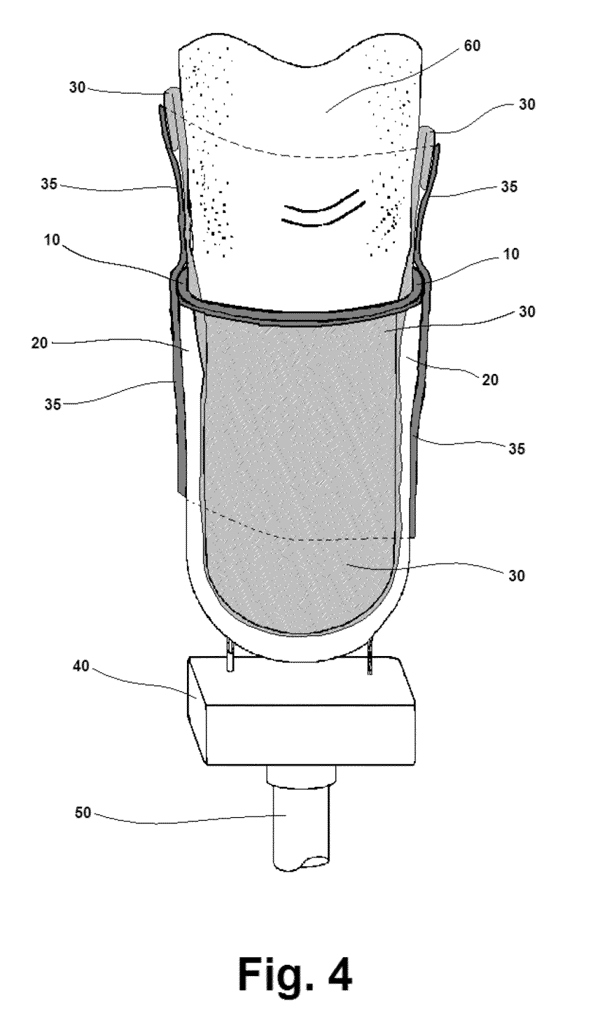

The sealing member 10 is located along the top edge 28 of the socket 20. See FIG. 3. It may be integrated therewith or separately applied and affixed thereto. In one embodiment the width of the sealing member 10 is substantially identical to the width of the top edge 28 of the socket 20. In another embodiment the width of the sealing member 10 is somewhat greater to the width of the top edge 28 of the socket 20, whereby at least a portion of the inner lateral side 16 of the sealing member 10 extends inward into the interior space 29 of the socket 20. In the preferred embodiment the outer lateral side 14 and the inner lateral side 16 of the sealing member 10 extend downward and over the outer surface 25 and inner surface 24, respectively, of the side wall 22 of the socket 20, proximate to its top edge 28. As shown in FIG. 4, the sealing member 10 provides a cushion between the top edge 28 of the socket 20 and the stump liner 30 when the top portions of the socket sleeve 35 and the stump liner 30 are folded down over the top edge 28 of the socket 20.

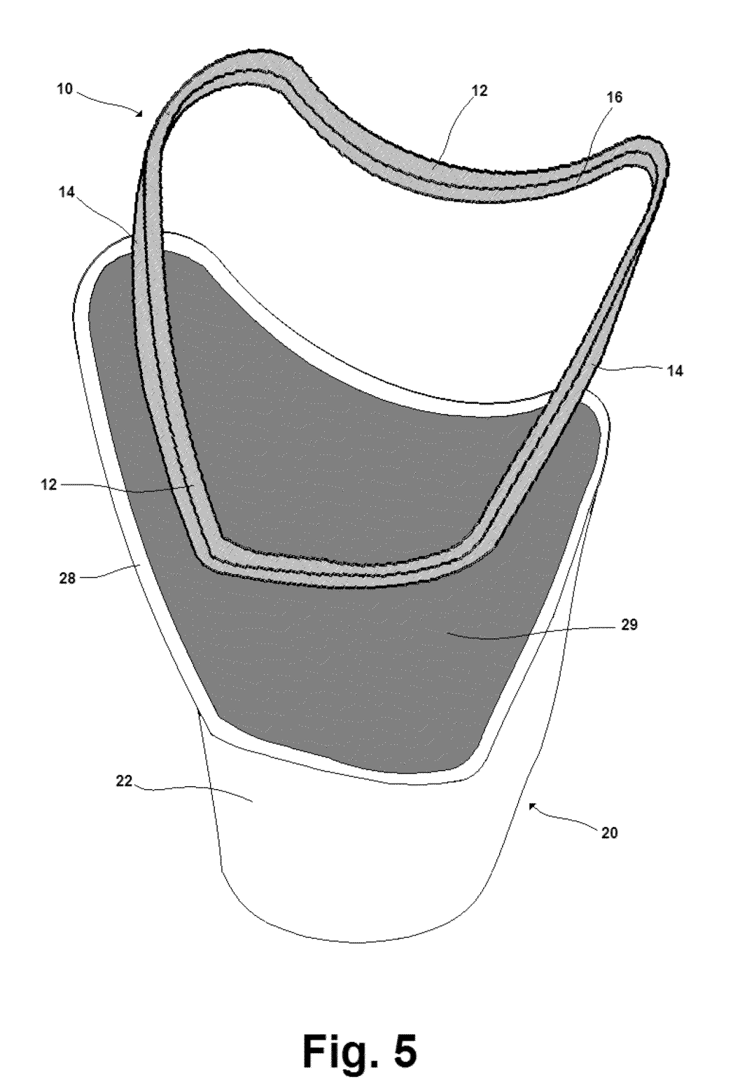

In one embodiment the sealing member 10 is pre-formed. See FIG. 5. In this embodiment it is configured as a substantially continuous ring, having a channel formed between its outer lateral side 14 and its inner lateral side 16. It is placed onto the socket 20 by inserting the top edge 28 of the socket 20 into the channel. It may be secured to the socket 20 by friction, or an adhesive may first be applied to the inside of the channel and/or the top edge 28 of the socket 20.

In another embodiment the sealing member 10 is formed onto the socket 20. In this embodiment the sealing member 10 is made of a material having a liquid state and a solid state. The sealing member 10 is applied to the top edge 28 of the socket 20 while the material is in its liquid state and then allowed to harden into its solid state. This process may be repeated multiple times to build up the height and width of the sealing member 10. In variants of this embodiment the sealing member 10 is applied to a portion of the outer surface 25 of the side wall 22 of the socket 20 adjacent to the top edge 28 of the socket 20 while the material is in its liquid state and then allowed to harden into its solid state. In other variants the sealing member 10 is applied to a portion of the inner surface 24 of the side wall 22 of the socket 20 adjacent to the top edge 28 of the socket 20 while the material is in its liquid state and then allowed to harden into its solid state. As before, either or both of these processes may be repeated multiple times to build up the height and width of the sealing member 10.

The present invention also contemplates a method for retrofitting a socket 20 used in a vacuum suspension system. The method comprises the steps of:

A. obtaining a material having a liquid state and a solid state;

B. applying the material to the top edge 28 of the socket 20 while the material is in its liquid state; and

C. allowing the material to harden into its solid state.

In this embodiment the sealing member 10 is made of a material having a liquid state and a solid state. An example of such a material is synthetic rubber, such as Performix® Brand Plasti Dip®. This process may be repeated multiple times to build up the height and width of the sealing member 10.

A variation of this method includes the following additional steps:

B1. applying the material to a portion of the outer surface 25 of the side wall 22 of the socket 20 adjacent to the top edge 28 of the socket 20 while the material is in its liquid state; and

B2. applying the material to a portion of the inner surface 24 of the side wall 22 of the socket 20 adjacent to the top edge 28 of the socket 20 while the material is in its liquid state.

Steps B1 and B2 may be performed in any order after step A and before step C. These steps allow for the sealing member 10 to better adhere to the socket 20.

Another variation of this method includes the following additional step:

A1. preparing the socket 20 to receive the material by sanding the top edge 28 of the socket 20.

Step A1 may be performed at any time before step B. Sanding is understood to mean not only the roughening of the surface of the top edge 28 of the socket 20 by the use of sand paper, but also a roughening of the surface of the top edge 28 of the socket 20 by the use of and suitable means, such as a wire brush or file.

Alternatively, the following steps may also be performed in conjunction with Step A1:

A2. preparing the socket 20 to receive the material by sanding the portion of the outer surface 25 of the side wall 22 of the socket 20 adjacent to the top edge 28 of the socket 20; and

A3. preparing the socket 20 to receive the material by sanding the portion of the inner surface 24 of the side wall 22 of the socket 20 adjacent to the top edge 28 of the socket 20.

Steps A1, A2, and A3 may be performed in any order and at any time before steps B, B1, B2, and C.

In another embodiment of the method for retrofitting a socket 20 used in a vacuum suspension system, the method comprises the steps of:

A. obtaining a pre-formed sealing member 10; and

B. placing the sealing member 10 onto the top edge 28 of the socket 20.

The pre-formed sealing member 10 is configured as a substantially continuous ring, having a channel formed between its outer lateral side 14 and its inner lateral side 16. See FIG. 5. It is placed onto the socket 20 by inserting the top edge 28 of the socket 20 into the channel.

A variation of this method includes the following additional step:

A1. placing an adhesive onto the top edge 28 of the socket 20.

Step A1 is performed before step B.

The adhesive may be any suitable adhesive capable of bonding rubberized and plastic materials.

Modifications and variations can be made to the disclosed embodiments of the sealing member 10 without departing from the subject or spirit of the invention as defined in the following claims.

I claim:

1. A method for retrofitting a socket used in a vacuum suspension system, wherein
the vacuum suspension system comprises the socket, a stump liner, and a vacuum pump,
with the socket being a rigid container having substantially continuous side and bottom walls and inner and outer surfaces and an open top end defined by a top edge, thereby forming an interior space, said interior space being configured to receive a residual stump of an amputee, said socket further providing an attachment means on its outer surface for securing a prosthetic leg thereto,
the stump liner being a flexible sleeve configured to fit snugly over the residual stump, the stump liner being substantially air impermeable, said stump liner being capable of forming an air impermeable barrier against the inner surface of the socket when worn over the residual stump and placed into the socket, and
the vacuum pump being in communication with the interior space of the socket and being capable of evacuating air from the interior space of the socket to create at least a partial vacuum therein;
said method comprising the steps of:
A. obtaining a material having a liquid state and a solid state;
B. applying the material to the top edge of the socket while the material is in its liquid state; and
C. allowing the material to harden into its solid state.

2. The method of claim 1 further comprising the following steps:
B1. applying the material to a portion of the outer surface of the side wall of the socket adjacent to the top edge of the socket while the material is in its liquid state; and
B2. applying the material to a portion of the inner surface of the side wall of the socket adjacent to the top edge of the socket while the material is in its liquid state;
whereby steps B1 and B2 may be performed in any order after step A and before step C.

3. The method of claim 1 wherein the material is synthetic rubber.

4. The method of claim 2 wherein the material is synthetic rubber.

5. The method of claim 1 further comprising the following step:
A1. preparing the socket to receive the material by sanding the top edge of the socket;
whereby step A1 may be performed at any time before step B.

6. The method of claim 2 further comprising the following steps:
A1. preparing the socket to receive the material by sanding the top edge of the socket;
A2. preparing the socket to receive the material by sanding the portion of the outer surface of the side wall of the socket adjacent to the top edge of the socket; and
A3. preparing the socket to receive the material by sanding the portion of the inner surface of the side wall of the socket adjacent to the top edge of the socket;
whereby steps A1, A2, and A3 may be performed in any order and at any time before steps B, B1, B2, and C.

7. The method of claim 5 wherein the material is synthetic rubber.

8. The method of claim 6 wherein the material is synthetic rubber.
